(19) United States
(12) Patent Application Publication
(10) Pub. No.: US 2006/0222595 A1
(43) Pub. Date: Oct. 5, 2006

(54) Nanoparticles for therapeutic and diagnostic applications

(76) Inventors: Priyabrata Mukherjee, Rochester, MN (US); Resham Bhattacharya, Rochester, MN (US); Debabrata Mukhopadhyay, Rochester, MN (US)

Correspondence Address: FISH & RICHARDSON P.C., PO BOX 1022, MINNEAPOLIS, MN, 55440-1022, US

(21) Appl. No.: 11/392,448

(22) Filed: Mar. 29, 2006

Related U.S. Application Data
(60) Provisional application No. 60/667,169, filed on Mar. 31, 2005.

(51) Int. Cl. A61K 49/00; A61K 39/395; A61K 9/14

(52) U.S. Cl. 424/9.34; 424/9.6; 424/178.1; 424/489; 977/906

(57) ABSTRACT

This document provides materials and methods related to nanoparticles. For example, nanoparticle compositions, methods for making nanoparticle compositions, and methods for using nanoparticle compositions are provided. In some cases, the nanoparticles are gold (e.g., colloidal gold) nanoparticles. A nanoparticle can include one or more agents linked to its surface, such as therapeutic and/or diagnostic agents, and can be from about 1 nm to about 10 nm in size.

### CROSS-REFERENCE TO RELATED APPLICATIONS

This application claims priority under 35 U.S.C. § 119 to U.S. Provisional Application Ser. No. 60/667,169, filed Mar. 31, 2005, incorporated by reference in its entirety herein.

### STATEMENT AS TO FEDERALLY FUNDED RESEARCH

Funding for the work described herein was provided in part by the federal government, which may have certain rights in the invention.

### BACKGROUND

1. Technical Field

This disclosure relates to nanoparticles, and more particularly to nanoparticles having one or more agents linked to their surfaces for diagnostic and therapeutic applications.

2. Background Information

A variety of therapeutic and diagnostic agents that have been isolated from natural sources or synthesized de novo demonstrate limited therapeutic or diagnostic efficacy, often due to inadequate delivery of the agent to a required site of action. For example, some agents, including some particulate agents, are actively removed from circulation by clearance mechanisms such as the reticuloendothelial system (RES). Other agents are unacceptably toxic when systemically administered.

### SUMMARY

This disclosure involves materials and methods for treating and/or diagnosing a variety of diseases, including cancer. For example, this disclosure provides compositions and kits that include nanoparticles having one or more agents, such as therapeutic or diagnostic agents, linked thereto. This disclosure also provides methods for treating a disease or ameliorating one or more symptoms of a disease using the described compositions, methods for preparing the compositions, methods for monitoring the efficacy of a therapy, and methods for imaging a disease state, such as a tumor, using the described compositions.

In some embodiments, the compositions and kits provided herein can be used to treat cancer in a patient. The cancer can be a solid tumor, e.g., a liver, kidney, stomach, gall bladder, lung, or spleen tumor. For example, a composition that includes nanoparticles having both an anti-VEGF antibody and a chemotherapeutic agent (e.g., gemcitabine) linked independently to the nanoparticle can be administered to a patient having a solid tumor to treat the solid tumor. In some cases, a composition containing nanoparticles having both an anti-VEGF antibody and a magnetic resonance contrast imaging agent linked to its surface can be used to treat cancer, to diagnose cancer (e.g., to image a tumor), and/or to monitor the efficacy of a treatment (e.g., to image a tumor at multiple timepoints during treatment).

Compositions provided herein can be prepared using simple protocols that provide significant flexibility in agent choice for inclusion, allowing the resulting compositions to be tailor-made for use in a variety of therapeutic and diagnostic applications. In some cases, two therapeutic agents can be linked to a nanoparticle. The two therapeutic agents can perform two different therapeutic functions in vivo. For example, one therapeutic agent can be a chemotherapeutic agent such as gemcitabine, while another therapeutic agent can be an anti-angiogenesis agent such as an anti-VEGF antibody. In some cases, a therapeutic agent and a diagnostic agent can be linked to the nanoparticle, facilitating the use of a composition in both diagnostic and therapeutic applications. In some cases, two diagnostic agents can be linked to the nanoparticle, e.g., to facilitate multiple modes of imaging or to provide increased signal sensitivity or resolution. Finally, compositions provided herein can be designed such that the nanoparticles lack a masking agent such as PEG Such compositions can avoid significant uptake and removal from circulation by the RES, thereby enhancing therapeutic or diagnostic efficacy through an extended circulation time.

In one aspect, a composition provided herein includes metal nanoparticles, where each of the metal nanoparticles is from about 1 nm to about 10 nm in size and includes at least one agent linked to its surface, and where at least about 30 percent of the metal nanoparticles of the composition lack PEG or a PEG-derivative linked to the surface of the nanoparticle. A metal nanoparticle can be from about 4 nm to about 6 nm. A metal nanoparticle can be gold, silver, copper, nickel, aluminum, zinc, calcium, platinum, palladium, or an iron nanoparticle. In some cases a metal nanoparticle is a gold nanoparticle.

An agent can be a therapeutic agent or a diagnostic agent. A therapeutic agent can be an anti-angiogenic agent, a chemotherapeutic agent, an anti-inflammatory agent, an anti-bacterial agent, an anti-fungal agent, a growth factor, an immunostimulatory agent, an anti-cholinergic agent, insulin, or an insulin analog.

An anti-angiogenic agent can be an anti-VEGF antibody, dopamine, an anti-endothelial adhesion receptor of integrin alpha v3 antibody, thalidomide, a thalidomide analog, a protein kinase C beta inhibitor, 2-methoxyestradiol, interferon alpha, or interleukin 12. A chemotherapeutic agent can be taxol, vinblastin, vincristine, acyclovir, tacrine, gemcitabine, paclitaxel, herceptin, methotrexate, cisplatin, bleomycin, doxorubicin, or cyclophosphamide.

A therapeutic agent can be in the form of an antibody, an antibody fragment, a receptor, a receptor fragment, a small-molecule, a peptide, a nucleic acid, or a peptide-nucleic acid.

A diagnostic agent can be an MR imaging agent, a radio-imaging agent, an X-ray imaging agent, or a near-IR imaging agent. An MR imaging agent can include a chelating ligand selected from the group consisting of DTPA, DOTA, DOTMA, DTPA-BMA, DOTAGA, and HP-DO3A.

In some embodiments a metal nanoparticle includes at least two agents linked to its surface. The two agents can be therapeutic agents. In some cases, one therapeutic agent is an anti-angiogenesis agent, such as an anti-VEGF antibody, and the other therapeutic agent is a chemotherapeutic agent, such as gemcitabine. In other cases, one agent is a diagnostic agent and the other is a therapeutic agent.

Pharmaceutically acceptable compositions are also provided herein and can include a composition described herein.

In another aspect, a method for treating or ameliorating one or more symptoms of cancer in a patient is provided. A method can include administering to the patient a pharmaceutically acceptable composition provided herein where the pharmaceutically acceptable composition includes a gold nanoparticle comprising an anti-angiogenesis agent linked to its surface. The gold nanoparticle can further include a chemotherapeutic agent linked to its surface. The anti-angiogenesis agent can be an anti-VEGF antibody, and the chemotherapeutic agent can be gemcitabine. The cancer can be a solid tumor, such as a renal carcinoma. The patient can be a mammal, such as a human, monkey, rat, mouse, cat, dog, sheep, pig, horse, or cow.

In another aspect, a method for inhibiting angiogenesis from a tumor in a patient is provided. The method can include administering a pharmaceutically acceptable composition provided herein to the patient where the pharmaceutically acceptable composition includes a gold nanoparticle comprising an anti-angiogenesis agent linked to its surface.

In yet another aspect, a method for imaging a tumor in a patient is provided which can include: administering a pharmaceutically acceptable composition provide herein to the patient, where the pharmaceutically acceptable composition includes a gold nanoparticle having an anti-VEGF antibody linked to its surface and a diagnostic agent linked to its surface; and imaging the patient. The diagnostic agent can be an MR imaging agent. The MR imaging agent can include a DTPA chelating ligand having Gd(III) coordinated thereto.

A method for reducing cell proliferation in a tumor in a patient is also provided and can include: administering a pharmaceutically acceptable composition provided herein to the patient, where the pharmaceutically acceptable composition includes a gold nanoparticle having a chemotherapeutic agent linked to its surface. Another method for reducing cell proliferation in a tumor in a patient can include: administering a pharmaceutically acceptable composition provided herein to the patient, where the pharmaceutically acceptable composition includes a gold nanoparticle having a anti-angiogenesis agent linked to its surface. A method for inducing apoptosis in a cell is also provided, wherein the cell is contacted with a composition comprising gold nanoparticle having an anti-angiogenesis agent linked to its surface.

In a further aspect, a method for monitoring whether or not a cancer therapy has had an effect on a tumor in a patient is provided. The method can include: a) administering a cancer therapy comprising a pharmaceutically acceptable composition provided herein to the patient, where the pharmaceutically acceptable composition includes a gold nanoparticle having a chemotherapeutic agent linked to its surface and a diagnostic agent linked to its surface; b) obtaining a first image of the tumor in the patient after the administration; c) obtaining a second image of the tumor in the patient after a pre-determined time period; and d) comparing the first and second images in order to determine whether or not the cancer therapy has affected the tumor.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1a is a graph plotting the change in absorbance of gold nanoparticle solutions upon linkage of the anti-VEGF antibody, followed by treatment with 10% NaCl.

FIG. 1b is a graph plotting the change in Ymax value of gold nanoparticle solutions upon linkage of the anti-VEGF antibody, followed by treatment with 10% NaCl.

FIG. 2 is a graph plotting the change in absorbance of gold nanoparticles upon linkage of gemcitabine.

FIG. 3a is a graph plotting the change in absorbance of gold nanoparticles upon linkage of gemcitabine and the anti-VEGF antibody.

FIG. 3b is a graph plotting the change in Ymax of gold nanoparticles upon linkage of both gemcitabine and the anti-VEGF antibody, followed by aggregation experiments with NaCl.

FIG. 4 demonstrates the efficacy of gemcitabine in the linked and unlinked form using a BrDu proliferation assay of 786-O cells.

FIG. 5 demonstrates the efficacy of the anti-VEGF antibody in the linked and unlinked form using an assay based on VEGF165-induced calcium release from HUVEC cells.

FIG. 6 is a graph plotting the saturation curve of dopamine on gold nanoparticles. The change in absorbance and shift in the Ymax value confirm the attachment of dopamine to the nanoparticle surfaces.

FIG. 7 are three graphs plotting the effect of conjugation of an anti-VEGF antibody (2C3) to gold nanoparticles (Gold-AVF) on apoptosis of three different CLL-B primary cells as compared to gold nanoparticles alone (Gold) or the anti-VEGF antibody (2C3) alone (not shown).

### DETAILED DESCRIPTION

This disclosure provides compositions and methods for diagnosing and treating a variety of diseases, including cancer. For example, this disclosure provides compositions that contain nanoparticles having at least one agent, such as a therapeutic or diagnostic agent, linked to the nanoparticle, e.g., linked to the surface of the nanoparticle. In some cases, two therapeutic agents can be linked to the nanoparticle, such as a chemotherapeutic and an anti-angiogenic agent. In some cases, the nanoparticles can provide a dual mechanism for treating cancer. In some cases, a nanoparticle can have a therapeutic and a diagnostic agent linked thereto. Such a nanoparticle can provide a means to both treat and to image cancer, thereby facilitating the ability to monitor the cancer therapy over time. Also provided herein are pharmaceutical compositions, kits, and methods for making and using the described nanoparticle compositions, e.g., to treat cancer and to inhibit angiogenesis.

Compositions

The compositions provided herein can contain nanoparticles having at least one agent linked thereto, e.g., linked to the surface of the nanoparticle. A composition typically includes many nanoparticles with each nanoparticle having at least one agent linked thereto. Nanoparticles can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl₄. Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl₄, are known to those having ordinary skill in the art. For example, the methods described herein as well as those described elsewhere (e.g., US 2001/005581; 2003/0118657; and 2003/0053983) can be used to make nanoparticles.

As indicated herein, a nanoparticle can have at least one agent linked to its surface. Any of the agents described herein can be linked covalently, noncovalently, or coordinately to the surface of the nanoparticle. For example, all the bonds from an agent to a nanoparticle can be covalent bonds to the surface of the nanoparticle. In some cases, some of the bonds are covalent to the surface of the nanoparticle, and some are noncovalent to the surface of the nanoparticle. In some cases, some of the bonds are covalent to the surface of the nanoparticle, and some are coordinate to the surface of the nanoparticle. In some cases, all of the bonds are noncovalent to the surface of the nanoparticle.

In certain cases, a nanoparticle can have two, three, four, five, six, or more agents linked to its surface. Typically, many molecules of an agent are linked to the surface of the nanoparticle at many locations. Accordingly, when a nanoparticle is described as having, for example, two agents linked to it, the nanoparticle has two distinct agents, each having its own unique molecular structure, linked to its surface. In some cases, one molecule of an agent can be linked to the nanoparticle via a single attachment site or via multiple attachment sites.

An agent can be linked directly or indirectly to a nanoparticle surface. For example, an agent can be linked directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In cases where the nanoparticle includes gold, a linker can be any thiol-containing molecule. Reaction of a thiol group with the gold results in a covalent sulfide (-S-) bond. Linker design and synthesis are well known in the art.

Any type of agent can be linked to a nanoparticle. For example, an agent can be a therapeutic agent that has a therapeutic effect in the body. Examples of a therapeutic agents include, without limitation, anti-angiogenic agents, chemotherapeutic agents, anti-inflammatory agents, anti-bacterial agents, anti-fungal agents, growth factors, immunostimulatory agents, anti-cholinergic agents, insulin, and insulin analogs.

An anti-angiogenic agent can be useful for inclusion on a nanoparticle in any disease state where it would be useful to prevent the formation of new blood vessels, e.g., to reduce tumor growth and metastasis. An anti-angiogenic agent can be any agent known to affect angiogenesis, and in certain cases can be an anti-VEGF antibody, dopamine, an anti-endothelial adhesion receptor of integrin alpha v3 antibody, thalidomide, a thalidomide analog, a protein kinase C beta inhibitor, 2-methoxyestradiol, interferon alpha, and interleukin 12.

In some cases, an anti-VEGF antibody, such as a monoclonal anti-VEGF antibody, can be used as an anti-angiogenic agent. While not being bound by any theory, it is believed that an anti-VEGF antibody can block the interaction of VEGF with blood vessel receptors, thereby inhibiting angiogenesis. Any anti-VEGF antibody can be used, including a monoclonal anti-VEGF antibody, an anti-VEGF antibody fragment, and a humanized version of an anti-VEGF antibody. Any method can be used to obtain such antibodies, including those described elsewhere (e.g., U.S. Pat. Nos. 6,344,339; 6,448,077; 6,676,941 and US 2003/0118657).

Any type of a chemotherapeutic agent can be linked to a nanoparticle, including for example, taxol, vinblastin, vincristine, acyclovir, tacrine, gemcitabine, paclitaxel, herceptin, methotrexate, cisplatin, bleomycin, doxorubicin, and cyclophosphamide. Any combinations of such chemotherapeutic agents can be used. Any method for preparing chemotherapeutic agents can be used, including those described elsewhere.

In some cases, a nanoparticle can have two therapeutic agents linked to it. For example, a nanoparticle can have both a chemotherapeutic agent and an anti-angiogenic agent linked thereto. In one embodiment, an anti-VEGF antibody and gemcitabine can be linked to a nanoparticle. In other embodiments, two different chemotherapeutic agents can be linked to a nanoparticle, such as paclitaxel and gemcitabine, or taxol and gemcitabine, or herceptin and gemcitabine. Such dual-therapeutic nanoparticles can exhibit increased efficacy as compared to each agent alone or each agent independently linked to a nanoparticle.

A therapeutic agent can be in any physical or chemical form, including an antibody, an antibody fragment, a receptor, a receptor fragment, a small-molecule, a peptide, a nucleic acid, and a peptide-nucleic acid.

A therapeutic agent can function as a targeting agent in addition to functioning as a therapeutic agent. A targeting functionality can allow nanoparticles to accumulate at the target at higher concentrations than in other tissues. In general, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function. Accordingly, an anti-VEGF antibody can target a nanoparticle to regions in the body where VEGF is produced or upregulated (e.g., tumors, blood vessels), while also exhibiting its therapeutic activity (e.g., reduction or inhibition of angiogenesis). In some cases, a receptor or receptor fragment can target a nanoparticle to a particular region of the body, e.g., the location of its binding pair member. Other therapeutic agents such as small molecules can similarly target a nanoparticle to a receptor, protein, or other binding site having affinity for the therapeutic agent.

A nanoparticle can have a diagnostic agent linked thereto. In some cases, a diagnostic agent and a therapeutic agent can both be linked to a nanoparticle. A diagnostic agent can allow the imaging of a nanoparticle in vivo. For example, a patient administered a nanoparticle having a diagnostic agent and a therapeutic agent linked thereto can be imaged once, e.g., to locate and/or stage a tumor, or at multiple time points, e.g., to monitor the efficacy of the therapeutic agent.

Any type of diagnostic agent can be linked to a nanoparticle, including, for example, an MR imaging agent, a radio-imaging agent, an X-ray imaging agent, and a near-IR imaging agent. Two or more diagnostic agents can also be linked to a nanoparticle, such as an MR imaging agent and an X-ray imaging agent, or a near-IR imaging agent and an MR imaging agent. An MR imaging agent can be a metal chelate, e.g., can include a chelating ligand and a paramagnetic metal ion coordinated thereto. Any type of chelating ligand can be used, including cyclic and acyclic chelating ligands such as DTPA, DOTA, DOTMA, DTPA-BMA, DOTAGA, and HP-DO3A. Examples of paramagnetic metal ions include, without limitation, Gd(III), Fe(III), Mn(II), Cr(III), Cu(II), Dy(III), Ho(III), Er(III), Eu(III), Tb(II), Tb(III), and Tb(IV).

One example of a nanoparticle having both a diagnostic agent and a therapeutic agent linked to its surface is a gold colloidal nanoparticle having a Gd(III)-DTPA diagnostic agent and an anti-angiogenic agent, such as dopamine or an anti-VEGF antibody, linked thereto. In some cases, a Gd(III)-DOTA diagnostic agent and a chemotherapeutic agent such as gemcitabine can be linked to a gold colloidal nanoparticle.

An agent can be linked to a nanoparticle using any method, including those described herein and elsewhere (e.g., U.S. 2001/0055581). Typically, a colloidal metal is incubated with the agent at a temperature from about 15 to about 50° C., e.g., about 20 to about 40° C., or about 24 to about 28° C. In some cases, an incubation can be done at about room temperature. An incubation period can range from about 5 minutes to about 24 hours, or any time therebetween, e.g., about 1 hour, 2 hours, 3 hours, 4 hours, 5 hours, 6 hours, 8 hours, 10 hours, 12 hours, 14 hours, 16 hours, 18 hours, 20 hours, 24 hours, or even longer. Additional considerations, such as pH, salt concentration, etc. can vary depending on the agent to be incorporated and the nature of the colloidal metal.

The concentration of agents can be adjusted so as to cover the entire surface or only a portion of the surface of the nanoparticle. If two or more agents are linked, they can be reacted (e.g., incubated), either sequentially or simultaneously, with the surface of the nanoparticle. In some cases, each agent is reacted at a concentration of about 50% or less than the saturation concentration of the agent for the nanoparticle. For purposes of this disclosure, the saturation concentration can be approximated as the concentration of agent at which a maximum of UV-Vis absorbance is obtained. In some cases, the concentration of the agents can be tailored according to the desired surface coverage. The resulting composition includes a nanoparticle having a surface that is linked to one or more agents. The population of the surface with such groups can be random or in a designed fashion.

A composition provided herein can include a masking group linked to the nanoparticle surface. A masking group is a moiety that is used to inhibit the uptake of the nanoparticles by clearance mechanisms such as the RES. Typically, masking groups include PEG, PEG-derivatives (e.g., methyl ester or ether derivatives), and gangliosides.

In some embodiments, compositions provided herein can contain nanoparticles that lack PEG or derivatized PEG groups linked to their surfaces. For example, 30, 40, 50, 60, 70, 80, 90, 95, or 100 percent of the nanoparticles of a composition can lack PEG and derivatized PEG groups linked to their surfaces. PEG and derivatized PEG groups have been used to inhibit the uptake of the nanoparticles by clearance mechanisms such as the RES. A PEG derivative can include ether and ester derivatives, such as methyl or ethyl ether or methyl or ethyl ester derivatives. A PEG or PEG derivative can include thiol derivatives for linking the PEG or PEG derivative to the nanoparticle. While not being bound by any theory, it is believed that the small size of the nanoparticles described herein (e.g., about 1 to about 10 nm) can allow the nanoparticles to exhibit a reduced or minimized clearance by the RES as compared to larger nanoparticles. The compositions provided herein can contain nanoparticles where at least 30 percent (at least 40, 50, 60, 70, 80, 90, 95, or 99 percent) of the nanoparticles of the composition are between about 1 to about 50 nm in diameter. For example, a composition provided herein can contain nanoparticles where at least 30 percent (at least 40, 50, 60, 70, 80, 90, 95, or 99 percent) of the nanoparticles of the composition are from about 1 to about 50 nm, from about 5 to about 30 nm, from about 15 to about 30 nm, from about 20 to about 30 nm, from about 10 to about 20 nm, from about 1 to about 30 nm, from about 1 to about 25 nm, from about 1 to about 20 nm, from about 1 to about 10 nm, from about 5 to about 25 nm, from about 5 to about 20 nm, from about 5 to about 15 nm, from about 5 to about 10 nm, from about 3 to about 8 nm, from about 2 to about 6 nm, or from about 4 to about 8 nm. Such a composition can be administered to a mammal and exhibit a reduced or minimized clearance by the RES as compared to larger nanoparticles (e.g., 200, 100, 50, or 30 nm nanoparticles).

Any of the compositions provided herein can be formulated to form a pharmaceutically acceptable composition adapted for human or animal patients. Pharmaceutically acceptable means that the composition can be administered to a patient or animal without unacceptable adverse effects. Pharmaceutically acceptable compositions include any pharmaceutically acceptable salt, ester, or other derivative that, upon administration, is capable of providing (directly or indirectly) a composition of the invention. Other derivatives are those that increase the bioavailability of the compositions when administered or which enhance delivery to a particular biological compartment.

Where necessary, the pharmaceutically acceptable compositions can include such ingredients as solubilizing agents, excipients, carriers, adjuvants, vehicles, preservatives, a local anesthetic, salts, flavorings, colorings, and the like. The ingredients may be supplied separately, e.g., in a kit, or mixed together in a unit dosage form. A kit can further include directions for administering the nanoparticle compositions and/or accessory items such as needles or syringes, etc.

Methods

This disclosure also provides methods for using the compositions provided herein. For example, a method for treating or ameliorating one or more symptoms of cancer in a patient can include administering to the patient a pharmaceutically acceptable composition described herein. A patient can be a human or animal patient, including a bird, dog, cat, mouse, rat, pig, cow, sheep, horse, or monkey. A pharmaceutically acceptable composition can include a nanoparticle having one (e.g., gemcitabine or an anti-VEGF antibody) or two (both gemcitabine and an anti-VEGF antibody) therapeutic agents linked thereto. In some cases, a pharmaceutically acceptable composition can include a nanoparticle having a diagnostic agent (e.g., an MR diagnostic imaging agent) and/or a therapeutic (e.g., a chemotherapeutic or anti-angiogenic agent) linked thereto.

The dosage to be administered and the mode of administration will depend on a variety of factors including age, weight, sex, condition of the patient, pharmacokinetic parameters of the composition, genetic factors, and the like. As one of skill in the art will recognize, a clinician will ultimately decide the dosage. A nanoparticle composition can be administered in any number of conventional ways, including orally, parenterally, and subcutaneously. Parenteral administration includes intravenous, intraarterial, interstitial, intrathecal, or intracavity administration.

In some cases, the cancer to be treated is a solid tumor, such as a lung, kidney, stomach, gall bladder, spleen, or liver tumor. For example, a renal carcinoma can be treated using the methods described herein. Efficacy of treatment can be monitored by evaluating the size of a tumor over the time course of treatment (e.g., with MRI or other diagnostic methods) or by monitoring for markers known to be associated with the cancer.

Compositions described herein can be used in a method for inhibiting angiogenesis, e.g., from a tumor, in a patient. In one embodiment, the method can include administering a pharmaceutical composition as described herein, where the pharmaceutically acceptable composition includes a nanoparticle having an anti-angiogenic agent, such as an anti-VEGF antibody, linked to its surface. In some cases, a nanoparticle can further include a chemotherapeutic agent such as gemcitabine, herceptin, or taxol linked to its surface. Inhibition of angiogenesis can be monitored in a variety of ways, including monitoring the size of a tumor, its blood volume, or monitoring for angiogenic markers such as VEGF.

The compositions provided herein can be used in other therapeutic areas. For example, the compositions can be used to reduce cell proliferation, e.g., in a tumor, in a patient. Such a method can include administering a pharmaceutically acceptable composition to the patient, where the pharmaceutically acceptable composition includes a nanoparticle having one or more chemotherapeutic agents linked thereto.

Some compositions described herein can be used to image a variety of disease states, including cancer. For example, a method for imaging a tumor in a patient can include (a) administering a pharmaceutically acceptable composition that includes a nanoparticle having a diagnostic agent linked thereto and (b) imaging the patient. Imaging can take place after an appropriate time period to allow accumulation of the composition at the target. In some cases, a nanoparticle can further include a therapeutic agent that also functions as a targeting agent, e.g., an antibody, to facilitate uptake of the nanoparticle at the tumor.

A diagnostic agent can be an MR imaging agent, such as Gd(III)-DTPA or Gd(III)-DOTA, as described herein. Such agents can be used in the same manner as conventional MRI contrast agents and can be useful for the diagnosis and staging of, e.g., solid lesions and tumors, such as liver, kidney, stomach, gall bladder, spleen, and lung tumors.

A nanoparticle composition can be useful for monitoring the efficacy of a treatment, such as a cancer treatment, over time. For example, a nanoparticle composition that includes an MR imaging agent linked to the nanoparticle and a therapeutic agent linked to the nanoparticle can be administered to a patient, either before or after positioning of the patient in an MR apparatus. The nanoparticle composition can be allowed to localize at one or more sites of interest before acquiring the MR images, and/or one or more MR images may be acquired during the localization phase. Any lesions or tumors can be identified by examining the image. Typically, the localization of the nanoparticles will appear bright in the image (i.e., positive contrast). Additional images can be obtained and examined after subsequent administrations of the nanoparticle compositions. The timing of subsequent administrations and imaging may be based on pre-determined time periods, e.g., schedules of chemotherapy. For example, a chemotherapeutic regimen may require administration of a nanoparticle composition as described herein once a week, once every two weeks, once a month, or once every two or three months. Such a pre-determined time period may depend on the nature of the chemotherapeutic agent attached to the nanoparticle. A subsequent image can then be obtained during a second or later administration of the nanoparticle composition. Two or more images can then be compared to determine whether or not the cancer therapy has affected the tumor, e.g., by reducing its size, volume, blood flow, blood volume, or density.

The invention will be further described in the following examples, which do not limit the scope of the invention described in the claims.

### EXAMPLES

Reagents: VEGF165 was obtained from R&D systems, Minneapolis, Minn. Gemcitabine was obtained as Gemzar from Eli Lilly and Company, Indianapolis, Ind. Tetrachloroauric acid trihydrate and sodium borohydride were purchased from Sigma-Aldrich, St. Louis, Mo. Monoclonal anti-VEGF antibody (2C3) was obtained from Pregerine Pharmaceuticals.

### Example 1

### Preparation of Gold Nanoparticles

In a typical experiment, 50 ml of an aqueous solution containing 4.3 mg of solid sodium borohydride was added to 100 ml of 100 μM aqueous solution of tetracholoroauric acid under vigorous stirring. After overnight stirring, the gold nanoparticles thus formed were filtered through a 0.22 μm filter.

### Example 2

### Determination of the Saturation Concentration of Individual Agents on Gold Nanoparticles

The individual saturation concentrations of a monoclonal anti-VEGF antibody agent and a gemcitabine agent to gold nanoparticles were determined in the following manner. Eight tubes containing 1 ml of gold nanoparticles each were incubated with increasing concentrations of the anti-VEGF antibody for 30 minutes, followed by an aggregation assay involving incubation with 100 μl of a 10% NaCl solution. Approximately 15 min. after the addition of the NaCl solution, the UV-Visible spectra were recorded using a Shimadzu model system (UV2401 PC), and the saturation concentration of the antibody was determined.

The attachment of gemcitabine to the gold nanoparticles and the determination of its saturation concentration were performed similarly.

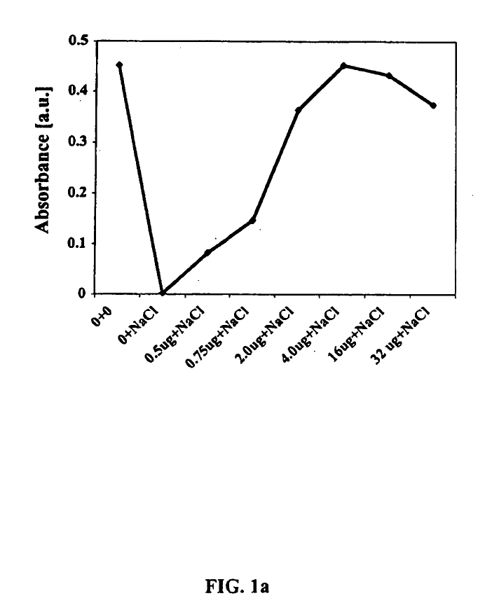

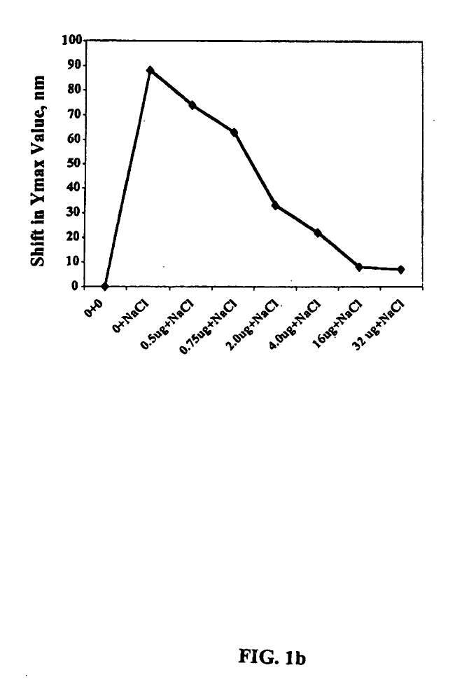

Results: With no agents linked to their surface, gold nanoparticles should aggregate immediately upon addition of a 10% NaCl solution and the extent of aggregation should decrease with increasing protection of the surface due to the linkage of one or more agents. Increased stabilization of the nanoparticle dispersions with increasing concentrations of the anti-VEGF antibody 2C3 was observed (FIG. 1a). For example, in the absence of antibody but in the presence of NaCl (0+NaCl), gold nanoparticles completely aggregated, and the UV-Vis absorbance dropped to zero. The extent of aggregation decreased with increasing concentrations of 2C3, and thus yielded an increase in absorbance (FIG. 1a). The absorbance reached a maximum at 4 μg antibody/ml of gold nanoparticle solution. The change in the Ymax value of the above samples (FIG. 1b) also exhibited a similar trend. The shift was maximal in the absence of any stabilizing antibody and gradually decreased with increasing concentrations of antibody. The nanoparticle composition was further characterized by transmission electron microscopy (TEM) after negative staining with uranyl acetate. Considering the change in absorbance with the addition of antibody and the shift in the Ymax value, it can be concluded that 4 μg anti-VEGF Ab/ml of nanoparticle gold solution is the saturation point.

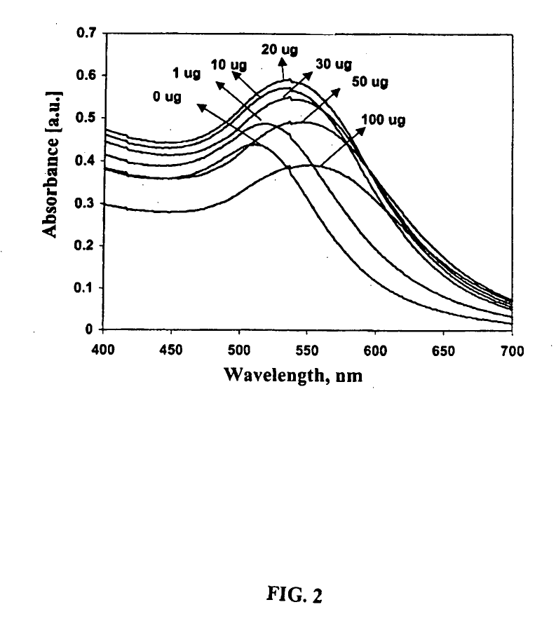

The saturation concentration of gemcitabine on the gold nanoparticles was determined in a similar manner. Gold nanoparticles were incubated with different concentrations of gemcitabine for 30 min., and their UV-Visible spectra were recorded. The absorbance of the solutions gradually increased with increasing concentrations of gemcitabine until the absorbance reached a maximum at 20 μg (FIG. 2). According to Mie theory, the observed shift in the Yₘₐₓ value with an increase in plasmon resonance coincides with a rising dielectric constant of the medium surrounding the gold nanoparticles; see, e.g., Kreibig, U., Vollmer, M. (1995), Optical properties of metal clusters; Springer Series in Material Science, 25; Springer-Verlag: Berlin. At doses higher than 20 μg, the absorbance of the nanocomposite gradually decreased, coupled with a gradual increase in red shift in the Yₘₐₓ value. This shift in Yₘₐₓ value with a concomitant decrease in absorbance and a broadening of the spectra may be attributed to the aggregation of gold nanoparticles upon the addition of gemcitabine beyond the 20 μg limit.

### Example 3

### Preparation of a Gold Nanoparticle Having Two Agents Linked Thereto

A composition having both gemcitabine and anti-VEGF antibody linked to the surface of gold nanoparticles was prepared by incubating gold nanoparticles with each agent at a concentration representing 50% of the saturation concentration of each individual agent. Thus, gold nanoparticles were first incubated for 30 min. at room temperature with 10 μg/ml of gemcitabine, followed by another 30 min. incubation with 2 μg/ml of the monoclonal anti-VEGF antibody. In order to confirm the linkage of both agents to the gold nanoparticles, aggregation experiments were performed by adding 100 μl of a 10% NaCl solution to solutions prepared as above, followed by incubation for 15 min. The UV-Visible spectra were subsequently recorded.

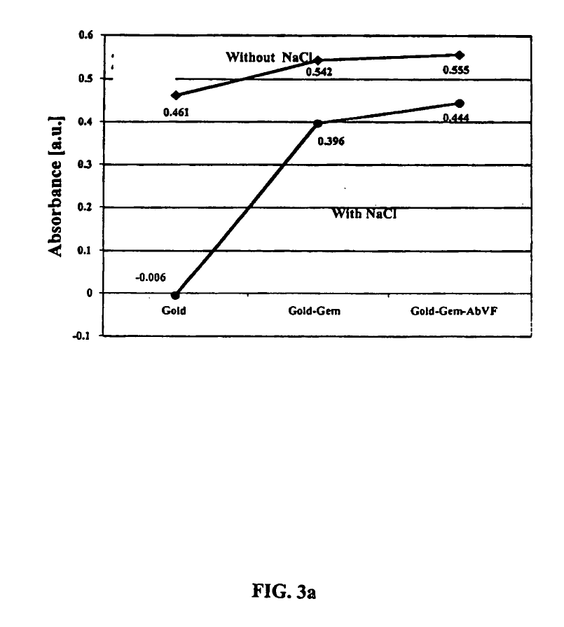

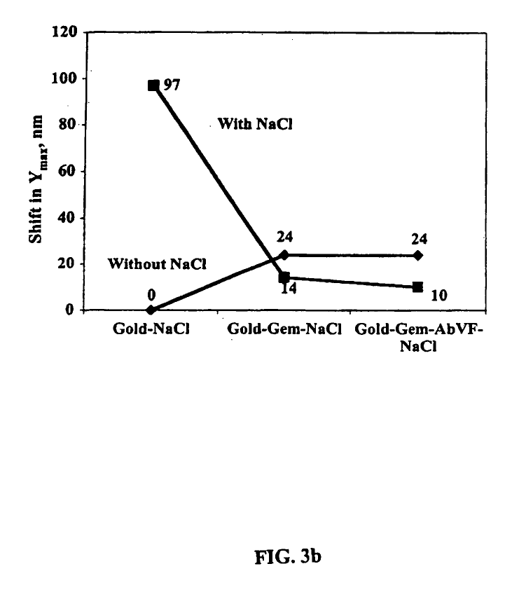

Results: A change in absorbance of the gold nanoparticles after incubation with both antibody and gemcitabine below their individual saturation points was observed (FIG. 3a). The results demonstrate better stabilization and increased absorbance of the gold nanoparticles with both the antibody and gemcitabine as compared to each component alone (FIG. 3a). This finding also confirms the attachment of both the components to the same nanoparticle surface. The shift in the Ymax value is minimal when the antibody and gemcitabine are both present on the nanoparticle surface as compared to each component alone (FIG. 3b). The minimum shift in the presence of both the antibody and gemcitabine confirms increased protection of the nanoparticle surfaces and suggests the successful attachment of both components.

### Example 4

### Determination of the Functional Activity of Agents Bound to Gold Nanoparticles

For the in vitro cell culture assays and calcium experiments described below, 50 ml of gold nanoparticles were first incubated with 500 μg of gemcitabine and 100 μg of the anti-VEGF antibody. The nanoparticle compositions were concentrated to one ml by ultracentrifugation at 30,000 rpm for 45 min. In proliferation experiments and calcium experiments, the concentrations of gemcitabine and anti-VEGF antibody were determined on the basis of the total amount of gemcitabine and antibody present in the nanoparticle composition, respectively.

The functional activities of nanoparticle-linked gemcitabine and nanoparticle-linked anti-VEGF antibody as compared to the unlinked agents were examined using in vitro cell culture assays. The activity of linked and unlinked gemcitabine was evaluated using a BrdU cell proliferation assay kit purchased from CALBIOCHEM and carried out on 786-O cells according the manufacturer's instructions (Cat. No. QIA58).

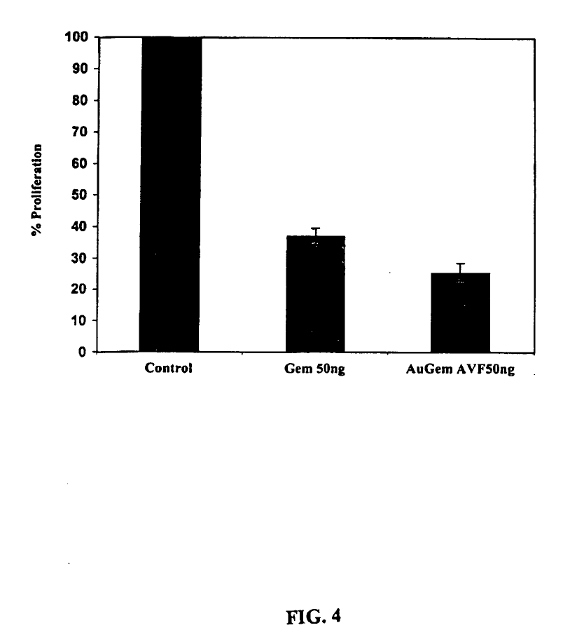

Results: The activity of gemcitabine bound to the nanoparticles was compared to the activity of gemcitabine alone (FIG. 4). The figure shows that the activity of gemcitabine on nanoparticles having both anti-VEGF antibody and gemcitabine bound is comparable to the activity of gemcitabine alone at the same concentration. These findings also confirm the linkage of gemcitabine on the gold nanoparticles. In control experiments with gold nanoparticles or the anti-VEGF antibody alone, no inhibition of 786-O proliferation was observed. The inhibition is therefore due to the presence of gemcitabine on the nanoparticles.

The effect of anti-VEGF antibody on VEGF165-induced calcium release in HUVEC cells was evaluated. HUVEC cells release calcium into the cytoplasm when induced with VEGF165. Serum starved HUVECs were loaded with Fura-2 AM dye and stimulated with 10 ng/ml VEGF 165, and then incubated with either anti-VEGF antibody or a composition having both gemcitabine and the anti-VEGF antibody linked to gold nanoparticles for 30 min. at 4° C. The experiments were carried out at least three times and as described elsewhere (see, e.g., Zeng, H., Zhao, D., and Mukhopadhyay, D. (2002), “KDR stimulates endothelial cell migration through heterotrimeric G protein Gq/11-mediated activation of a small GTPase RhoA,” J. Biol. Chem. 277: 4003-4009.)

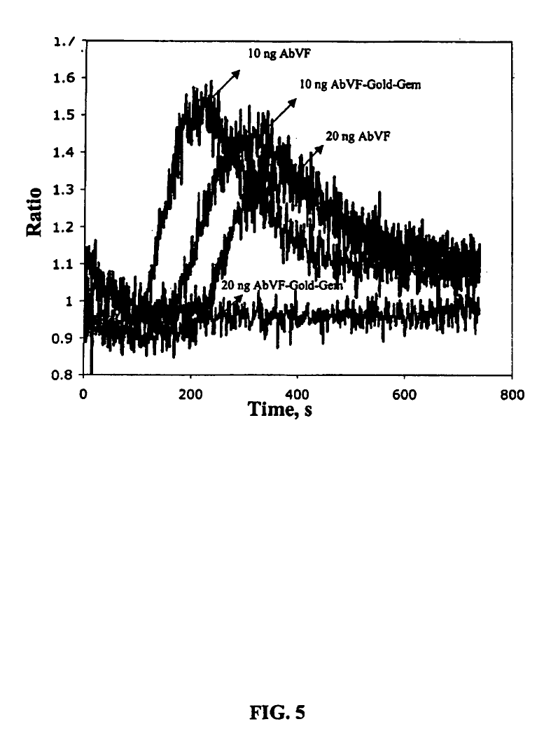

Results: The compositions having gemcitabine and the anti-VEGF antibody linked to gold nanoparticles inhibited calcium release more efficiently than the same dose of anti-VEGF antibody alone (FIG. 5). At 10 ng concentrations, almost no inhibition of VEGF165-induced calcium release was observed in the samples of antibody alone, but a moderate inhibition and delay was observed with the nanoparticles. At 20 ng concentrations, a moderate inhibition was observed in samples with only unlinked antibody, but a complete inhibition of calcium release was observed for the nanoparticles. To confirm that the inhibition in calcium release was due to the presence of the antibody on the nanoparticles, a nanoparticle surface was blocked with gemcitabine and IgG, and the experiment repeated. No inhibition of VEGF165-induced calcium release was observed with such control nanoparticles. These results indicate that the anti-VEGF antibody retains its functional activity when it is linked to the nanoparticle.

### Example 5

### Preparation of Nanoparticles Having a Therapeutic and Diagnostic Agent Linked Thereto

Dopamine, an anti-angiogenic therapeutic agent, and gado-diamide, an MRI diagnostic imaging agent, were conjugated to gold nanoparticles as described below.

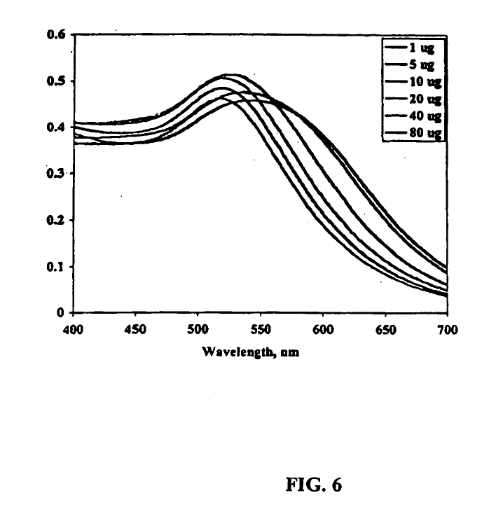

Saturation curves of dopamine on gold nanoparticles were prepared by adding varying amounts of dopamine to several vials containing 1 ml of gold nanoparticles prepared as described above, followed by measurement of the surface plasmon resonance band using UV-visible spectroscopy. The saturation curve of dopamine on gold nanoparticles was obtained (FIG. 6). The change in absorbance and shift in the Ymax value confirm the attachment of dopamine to nanoparticle surfaces. The saturation concentration was ˜30 μg/ml.

Gado-diamide was then attached to the gold nanoparticles already linked to dopamine by stirring the dopamine-gold nanoparticles with a gado-diamide solution for 12 hours. After 12 hours, the nanoparticles were purified by ultracentrifugation followed by washing twice with distilled water. The nanoparticles thus obtained were evaluated for Gd content using MRI. The attachment of gado-diamide to the gold-dopamine nanoparticles was observed.

### Example 6

### Evaluation of RES Uptake

A series of gold nanoparticle solutions that include, independently, gold nanoparticles ranging in size from about 1 to about 50 nm are prepared and evaluated for their ability to avoid or to limit RES uptake. RES uptake is evaluated by comparing the biodistribution profile of nanoparticles after administration to a test animal such as a mouse (e.g., by comparing biodistribution profiles in one or more of the liver, kidney, spleen, and blood versus a target organ such as a tumor). Biodistribution profile analyses are performed as described elsewhere (see, e.g., US 2003/0053983). The nanoparticle solutions are prepared as described herein and elsewhere (see, e.g., U.S. 2001/005581; 2003/0118657; and 2003/0053983). Briefly, a series of compositions that contain gold nanoparticles having an anti-VEGF antibody linked to the gold nanoparticle surfaces are prepared. The series is designed to include gold nanoparticles having a size, independently, of about 50, 45, 40, 35, 30, 25, 20, 15, 10, and 5 nm. The RES uptake and/or target uptake of such a series is evaluated and compared to a similar series having PEG linked to the surface of the gold nanoparticles in addition to the anti-VEGF antibody.

### Example 7

### Induction of Apoptosis by Anti-VEGF-Antibody Conjugated Gold Nanoparticles

This example demonstrates that gold-AbVEGF nanoconjugates are more efficient in the induction of apoptosis in B-CLL primary cells when compared to AbVEGF alone or gold nanoparticles alone.

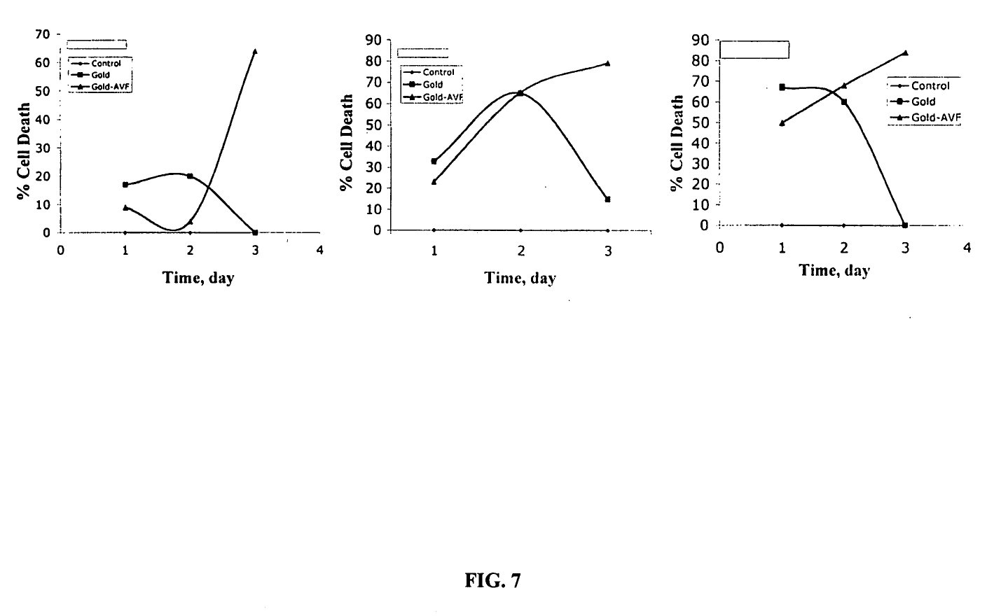

To compare nanoparticles conjugated with AbVEGF (gold-2C3) to AbVEGF alone, a humanized anti-VEGF-A monoclonal antibody 2C3 (Pergerine Pharma) was used in co-culture with purified CLL B primary cells. In brief, CLL B cells (1×10⁶) from three different CLL-B primary cells were co-cultured with increasing concentrations of 2C3 conjugated to gold nanoparticles, 2C3 alone, and gold nanoparticles alone (1 mg-25 mg/ml) for 24-72 hours. Annexin/PI flow cytometry was performed at varying time periods (e.g., 24, 48, and 72 hours) on the cells. The data demonstrate that conjugation to gold nanoparticles increases the efficacy of the antibody in inducing apoptosis (FIG. 7). Some degree of apoptosis by gold nanoparticles alone was also observed. Apoptosis by antibody alone remained about 10-15% (data not shown).

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A composition consisting of gold nanoparticles, wherein each of said gold nanoparticles is from about 4 nm to about 6 nm in size and comprises, linked to its surface, (a) at least one anti-VEGF antibody, (b) at least one molecule of gemcitabine, (c) at least one molecule of Gd(III)-DOTA, and wherein at least about 99 percent of the gold nanoparticles of said composition lack PEG or a PEG-derivative linked to their surfaces.

2. A composition comprising metal nanoparticles, wherein each of the metal nanoparticles is from about 1 nm to about 10 nm in size, and wherein each metal nanoparticle includes at least one agent linked to its surface, and wherein at least about 30 percent of the metal nanoparticles of the composition lack PEG or a PEG-derivative linked to the surface of the nanoparticle.

3. The composition of claim 2, wherein the metal nanoparticle can be from about 4 nm to about 6 nm.

4. The composition of claim 2, wherein the metal nanoparticle can be gold, silver, copper, nickel, aluminum, zinc, calcium, platinum, palladium, or an iron nanoparticle.

5. The composition of claim 4, wherein the metal nanoparticle is a gold nanoparticle.

6. The composition of claim 2, wherein the agent is a therapeutic agent or a diagnostic agent.

7. The composition of claim 6, wherein the therapeutic agent can be an anti-angiogenic agent, a chemotherapeutic agent, an anti-inflammatory agent, an anti-bacterial agent, an anti-fungal agent, a growth factor, an immunostimulatory agent, an anti-cholinergic agent, insulin, or an insulin analog.

8. The composition of claim 7, wherein the anti-angiogenic agent can be an anti-VEGF antibody, dopamine, an anti-endothelial adhesion receptor of integrin alpha v3 antibody, thalidomide, a thalidomide analog, a protein kinase C beta inhibitor, 2-methoxyestradiol, interferon alpha, or interleukin 12.

9. The composition of claim 7, wherein the chemotherapeutic agent can be taxol, vinblastin, vincristine, acyclovir, tacrine, gemcitabine, paclitaxel, herceptin, methotrexate, cisplatin, bleomycin, doxorubicin, or cyclophosphamide.

10. The composition of claim 6, wherein the therapeutic agent can be in the form of an antibody, an antibody fragment, a receptor, a receptor fragment, a small-molecule, a peptide, a nucleic acid, or a peptide-nucleic acid.

11. The composition of claim 6, wherein the diagnostic agent can be an MR imaging agent, a radio-imaging agent, an X-ray imaging agent, or a near-IR imaging agent.

12. The composition of claim 11, wherein the MR imaging agent can include a chelating ligand selected from the group consisting of DTPA, DOTA, DOTMA, DTPA-BMA, DOTAGA, and HP-DO3A.

13. The composition of claim 2, wherein the metal nanoparticle includes at least two agents linked to its surface.

14. The composition of claim 13, wherein the at least two agents are therapeutic agents.

15. The composition of claim 14, wherein one therapeutic agent is an anti-angiogenesis agent and the other therapeutic agent is a chemotherapeutic agent.

16. The composition of claim 13, wherein one agent is a diagnostic agent and the other agent is a therapeutic agent.

17. A pharmaceutically acceptable composition comprising the composition of claim 2 and a pharmaceutically acceptable carrier or diluent.

18. A method for treating or ameliorating one or more symptoms of cancer in a patient, the method comprising administering to the patient a pharmaceutically acceptable composition comprising a gold nanoparticle having an anti-angiogenesis agent linked to its surface.

19. The method of claim 18, wherein the gold nanoparticle further includes a chemotherapeutic agent linked to its surface.

20. The method of claim 19, wherein the anti-angiogenesis agent is an anti-VEGF antibody, and the chemotherapeutic agent is gemcitabine.

21. The method of claim 18, wherein the cancer is a solid tumor.

22. The method of claim 21, wherein the solid tumor is a renal carcinoma.

23. The method of claim 18, wherein the patient is a mammal,

24. The method of claim 23, wherein the mammal is a human.

25. A method for inhibiting angiogenesis from a tumor in a patient, the method comprising administering to the patient a pharmaceutically acceptable composition comprising a gold nanoparticle comprising an anti-angiogenesis agent linked to its surface.

26. A method for imaging a tumor in a patient, the method comprising:
a) administering to the patient a pharmaceutically acceptable composition comprising a gold nanoparticle having an anti-VEGF antibody linked to its surface and a diagnostic agent linked to its surface; and
b) imaging the patient.

27. The method of claim 26, wherein the diagnostic agent is an MR imaging agent.

28. The method of claim 27, wherein the MR imaging agent comprises a DTPA chelating ligand having Gd(III) coordinated thereto.

29. A method for reducing cell proliferation in a tumor in a patient, the method comprising:
a) administering to the patient a pharmaceutically acceptable composition comprising a gold nanoparticle having a chemotherapeutic agent linked to its surface.

30. A method for reducing cell proliferation in a tumor in a patient, the method comprising:
a) administering to the patient a pharmaceutically acceptable composition comprising a gold nanoparticle having an anti-angiogenesis agent linked to its surface.

31. A method for inducing apoptosis in a cell, the method comprising:
a) contacting the cell with a composition comprising a gold nanoparticle having an anti-angiogenesis agent linked to its surface.

32. A method for monitoring whether or not a cancer therapy has had an effect on a tumor in a patient, the method comprising:
a) administering to the patient a cancer therapy comprising a pharmaceutically acceptable composition comprising a gold nanoparticle having a chemotherapeutic agent linked to its surface and a diagnostic agent linked to its surface;
b) obtaining a first image of the tumor in the patient after the administration;
c) obtaining a second image of the tumor in the patient after a pre-determined time period; and
d) comparing the first and second images in order to determine whether or not the cancer therapy has affected the tumor.
